(19) United States
(12) Patent Application Publication
(10) Pub. No.: US 2007/0088104 A1
(43) Pub. Date: Apr. 19, 2007

(54) Temperature regulating gel and article comprising the same

(75) Inventors: Yu-Chun Hung, Taoyuan Hsien (TW); Chi-Shu Wei, Taipei Hsien (TW); Chen-An Ku, Taipei City (TW); Yen-Hsi Lin, Taipei City (TW)

Correspondence Address: MARSHALL, GERSTEIN & BORUN LLP, 233 S. WACKER DRIVE, SUITE 6300, SEARS TOWER, CHICAGO, IL, 60606, US

(73) Assignee: TAIWAN TEXTILE RESEARCH INSTITUTE, Tu-Chen City (TW)

(21) Appl. No.: 11/546,468

(22) Filed: Oct. 11, 2006

(30) Foreign Application Priority Data
Oct. 19, 2005 (TW) TW 94136576

(51) Int. Cl. C08K 5/05; C07K 16/00; C07K 17/00

(52) U.S. Cl. 524/31; 524/556; 524/319; 524/379; 524/386; 530/300; 530/350

(57) ABSTRACT

A temperature regulating gel includes a hydrophilic polymeric substrate and a heat-storing material. The hydrophilic polymeric substrate may include a natural hydrogel, a synthetic hydrogel, or any combination thereof. The heat-storing material may include a phase change material, a plastic crystal, or any combination thereof. The present invention also discloses a temperature regulating article including the temperature regulating gel.

### RELATED APPLICATIONS

The present application is based on, and claims priority from, Taiwan Application Serial Number 94136576, filed Oct. 19, 2005, the disclosure of which is hereby incorporated by reference herein in its entirety.

### BACKGROUND

1. Field of Invention

The present invention relates to a temperature regulating device. More particularly, the present invention relates to a temperature regulating gel and a long-lasting temperature regulating article comprising the temperature regulating gel.

2. Description of Related Art

A lot of devices have been invented to regulate body temperature in different conditions. For example, heat bag containing iron powder, water and salt releases heat to maintain body temperature when user is feeling cold, or ice bag can be put on the injured muscle to reduce pain.

Without the aid of other temperature controlling devices, conventional temperature regulating devices cannot be long-lasting and temperature change in the beginning of temperature regulation could be too drastic. If additional temperature controlling device is used to improve temperature regulating performance, the additional weight of the temperature controlling device decreases the convenience thereof. In addition, most of these conventional devices are one-time-use products which contradict to waste avoidance and resource recovery.

### SUMMARY

A temperature regulating gel is provided. The temperature regulating gel contains hydrophilic polymeric substrate and heat-storing material. The hydrophilic polymeric substrate includes natural hydrogel, synthetic hydrogel, or any combination thereof. The heat-storing material includes phase change material, plastic crystal, or any combination thereof. The temperature regulating gel made of hydrophilic polymeric substrate and heat-storing material contains 30-90% water by weight. The combination of hydrophilic polymeric substrate and heat-storing material significantly improves durability of the temperature regulating gel.

An article comprising the temperature regulating gel is also provided. The article comprises a bottom layer, a gel layer, and a top layer. The gel layer is between the bottom layer and the top layer. The gel layer is made of the temperature regulating gel mentioned above.

According to an embodiment of the present invention, the natural gel in the hydrophilic polymeric substrate mentioned above can be, for example, anionic polymer, cationic polymer, amphoteric polymer, neutral polymer, or any combination thereof. Anionic polymer can be hyaluronic acid, alginic acid, alginic acid derivative, pectin, chondroitin sulfate, gum arabic, carrageenan, lakh, xanthan gum, cellulose and cellulose derivative, or any combination thereof. Cationic polymer can be chitosan, polylysine, or any combination thereof. Amphoteric polymer can be collagen, gelatin, fibrin, or any combination thereof. Neutral polymercan can be dextran, agarose, starch and starch derivative, or any combination thereof.

Furthermore, the above mentioned synthetic hydrogel can be non-cross-linked or cross-linked polymer such as, for example, polyethylene glycol and polyethylene glycol derivative, poly(acrylic acid), poly(acrylic acid) derivative, polyacrylamide, polyethacylamide, polyethacylamide derivative, polyvinylpyrrolidone, polyvinylpyrrolidone derivative, cellulose, cellulose derivative, silicone, silicone derivative, inorganic silicate, inorganic silicate derivative, magnesium aluminum silicate, modified natural hydrogel, or any copolymer of combination thereof.

The above mentioned phase change material can be, for example, hydrocarbon, hydrate, wax, oil, fatty acid, fatty acid ester, carboxylic acid ester, dibasic esters, 1-halide, primary alcohol, aromatic compound, clathrate compound, semi-clathrate compound, gaseous clathrae compound, stearic anhydride, ethylene carbonate, polyol, polymer, metal, or any combination thereof.

The above mentioned plastic crystal can be neopentyl glycol or its derivatives.

According to another embodiment of the present invention, the heat-storing material can be in the form of microcapsule dispersed in the temperature regulating gel. Furthermore, the temperature regulating gel comprises additives such as, medicine, cosmetic material, dye, pigment, enzyme, spice, moisturizer, preservative, bactericide, bacteriostat, excipient, or any combination thereof.

According to another embodiment of the present invention, the bottom layer or the top layer of the above mentioned temperature regulating article comprises fabric made of synthetic fiber such as, polyurethane, polyethylene, polypropylene, polyester, or Teflon, nonwoven fabric, synthetic leather, and animal leather.

According to another embodiment of the present invention, the above temperature regulating article comprises, at least, an adhesive layer between the bottom layer and the gel layer, or the adhesive layer can be below the bottom layer so the article can be adhered on user body. In addition, the bottom layer and the top layer can also be pocket-like in shape to contain the gel layer in the pocket.

The temperature regulating article comprises the temperature regulating gel with long-last and stable temperature regulation. The temperature regulating article is portable and no additional device is required. Furthermore, the temperature regulating gel and temperature regulating article is convenient, reusable and environmental protecting.

The temperature regulating gel of the invention has multiple industrial applications such as textile industrial, biomedical industrial, medical instrument industry, and cosmetic industry. The long-lasting temperature regulating gel can be essential material for moisturizing and temperature cooling applications such as wound cover and facemask. Furthermore, applications can be extended to health caring textile after combining with heat-storing material, or extended to other health caring products such as cosmetics and medical dressing after combining with Chinese herbal extracts.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is cross sectional diagram of a temperature regulating article of one embodiment of the present invention;

FIG. 2 is cross sectional diagram of a temperature regulating article of another embodiment of the present invention;

FIG. 3 is cross sectional diagram of a temperature regulating article of another embodiment of the present invention; and

FIGS. 4-7 are comparative results of thermal buffer tests for sample 1-4.

### DETAILED DESCRIPTION

Reference will now be made in detail to the present preferred embodiments of the invention, examples of which are illustrated in the accompanying drawings. Wherever possible, the same reference numbers are used in the drawings and the description to refer to the same or like parts.

A temperature regulating article comprising a temperature regulating gel with long-lasting and stable temperature regulation is provided. The temperature regulating article comprising temperature regulating gel has long-lasting and stable temperature regulation. The temperature regulating article is portable, reusable and environmental protecting.

A temperature regulating gel is provided. The temperature regulating gel is made of hydrophilic polymeric substrate, heat-storing material and proper amount of water. The heat-storing material is, at least, 0.5% by weight, water is about 30-90% by weight. The hydrophilic polymeric substrate is used to fix water molecules in the temperature regulating gel and the heat-storing material is used as a buffer to store heat and stabilize temperature change.

The above mentioned hydrophilic polymeric substrate can be natural hydrogel, synthetic hydrogel or any combination thereof. They typical natural hyudrogel can be anionic polymer, cationic polymer, amphoteric polymer, neutral polymer, or any combination thereof. Anionic polymer can be hyaluronic acid, alginic acid, alginic acid derivative, pectin, chondroitin sulfate, gum arabic, carrageenan, lakh, xanthan gum, cellulose and cellulose derivative, or any combination thereof. Cationic polymer can be chitosan, polylysine, or any combination thereof. Amphoteric polymer can be collagen, gelatin, fibrin, or any combination thereof. Neutral polymer can be dextran, agarose, starch and starch derivative, or any combination thereof. Furthermore, the above mentioned synthetic hydrogel can be non-cross-linked or cross-linked polymer such as, for example, polyethylene glycol and polyethylene glycol derivative, poly(acrylic acid), poly(acrylic acid) derivative, polyacrylamide, polyethacylamide, polyethacylamide derivative, polyvinylpyrrolidone, polyvinylpyrrolidone derivative, cellulose, cellulose derivative, silicone, silicone derivative, inorganic silicate, inorganic silicate derivative, magnesium aluminum silicate, modified natural hydrogel, or any copolymer of combination thereof. The above mentioned synthetic hydrogel is preferred to have high water content but still its shape can be fixed.

The above mentioned heat-storing material comprises phase change material, plastic crystal, or any combination thereof. The material with heat-storing property can be combined with hydrophilic polymeric substrate to prolong temperature regulation.

The above mentioned phase change material can be, for example, hydrocarbon, hydrate, wax, oil, fatty acid, fatty acid ester, carboxylic acid ester, dibasic esters, 1-halide, primary alcohol, aromatic compound, clathrate compound, semi-clathrate compound, gaseous clathrae compound, stearic anhydride, ethylene carbonate, polyol, polymer, metal, or any combination thereof. Please refer to patent WO 02/24992 A1 for detailed examples of each phase change material mentioned above (pp. 12-16). The WO 02/24992 A1 is entirely incorporated here by reference.

The plastic crystal is, for example, neopentyl glycol and its derivatives. For example, neopentyl glycol can store and dissipate heat at constant temperature by molecular isomerization through solid-solid phase transformation instead of conventional solid-liquid phase transformation. The solid-solid phase transformation occurs at 44° C. The better plastic crystal includes 2,2,-dimethyl-1,3-propylene glycol, and 2-hydroxymethyl-2 methyl-propanol.

According to another embodiment of the present invention, the heat-storing material can be in the form of microcapsule dispersed in the temperature regulating gel. Furthermore, the temperature regulating gel further comprises additives such as, medicine, cosmetic material, dye, pigment, enzyme, spice, moisturizer, preservative, bactericide, bacteriostat, excipient, or any combination thereof. The above mentioned excipient can be multi-valent metallic salt or its compound (for example, Mg, Ca, Pb, Zn, Cd, Al, Ti, Mn, Co, Ni, other multi-valent metallic salt, or a compound thereof), water, solvent (for example, alcohol or polyol), surfactants (for example, non-ionic, anionic, cationic, or amphoteric ionic surfactant), thickner (for example, sodium chloride, ammonium chloride, ethylene glycol stearate, natural hydrogel, synthetic hydrogel. The amount of excipient additive can modify the hardness of the temperature regulating gel.

When long-lasting temperature regulating gel is close to a heat source, temperature regulating gel absorbs heat and reaches an equilibrium temperature, then the heat-storing material continues absorbing heat from the temperature regulating gel but maintained at the same equilibrium temperature. After the absorbed heat reaches the heat capacity of the heat-storing material, temperature will be increased again and water in the temperature regulating gel will be evaporated, meanwhile, the heat stored in the heat-storing material of the temperature regulating gel will be dissipated while water is evaporating. Therefore, temperature of the temperature regulating gel will be cooled down so the temperature regulation can be prolonged.

When manufacturing the temperature regulating gel, temperature regulating gel precursor containing proper amount of hydrophilic polymeric substrate and water can be prepared in advanced, then mixed up with heat-storing material. If the heat-storing material is in the form of microcapsule, or dispersed in the temperature regulating gel precursor, a long-lasting temperature regulating gel according to the embodiment of the invention can be obtained. The temperature regulating ability and temperature regulating durability are decided by the type and amount of hydrophilic polymeric substrate and heat-storing material, as well as the volume of the temperature regulating gel.

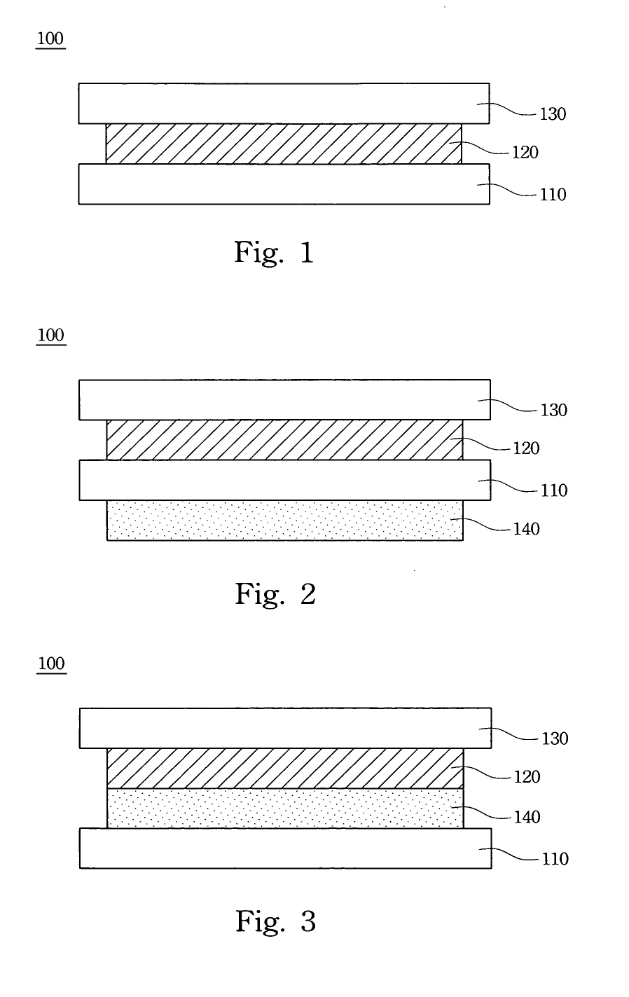

A temperature regulating article comprising the temperature regulating gel is introduced as follow. Please refer to FIG. 1, FIG. 1 is a cross-sectional diagram of a temperature regulating article according to one embodiment of the present invention. The temperature regulating article 100 comprises a bottom layer 110, a gel layer 120, and a top layer 130. The gel layer 120 is between the bottom layer 110 and the top layer 130. However, the bottom layer 110 can be removed and only the gel layer 120 and the top layer 130 are used, or the top layer 130 can be removed and only the bottom layer 110 and the gel layer 120 are used for temperature regulation. In addition, part of the surrounding area of the bottom layer 110 and the top layer 130 can be sealed so a pocket like structure can be formed to have the gel layer 120 contained in the pocket. Therefore, the gel layer 120 can be easily renewed.

The above-mentioned top layer 130 and the bottom layer 110 can be thin film, woven fabric, nonwoven fabric, synthetic leather, animal leather, polyurethane, polyethylene, polypropylene, polyester, Teflon, or any combination thereof.

The above mentioned top layer 130 and the bottom layer 110 can also be thermal conducting material such as metal or metallic composite material, non-metal or non-metal composite material, or any combination thereof. The metal can be Al, Ag, Ti, Cu, Au, Sn, stainless steel, or Pb. The non-metal can be graphite, quartz, borosilicate glass, or polymer.

Please refer to FIG. 2, FIG. 2 is a cross-sectional diagram of a temperature regulating article of another embodiment of the present invention. In FIG. 2, in addition to the bottom layer 110, the gel layer 120, and the top layer 130 as showed in FIG. 1, there is an adhesive layer 140 below the bottom layer 110 in order to allow the temperature regulating article 100 to be attached on an object, for example, to be attached on user's skin. The top layer 130 can be removed and only the gel layer 120, the bottom layer 110, and the adhesive layer 140 are used for temperature regulation.

Please refer to FIG. 3, FIG. 3 is cross sectional diagram of a temperature regulating article of another embodiment of the present invention. In addition to the bottom layer 110, the gel layer 120, and the top layer 130 as shown in FIG. 1, there is an adhesive layer 140 between the bottom layer 110 and the gel layer 120 to have the gel layer 120 attached to the bottom layer 110. The top layer 130 can be removed and only the gel layer 120, the adhesive layer 140 and the bottom layer 110 are used for temperature regulation.

To prevent water in the gel layer 120 from being over evaporated, the temperature regulating article 100 can be preserved in a sealed container before the use of the temperature regulating article 100. The temperature regulating article 100 can be taken out from the container right before using the temperature regulating article 100 to ensure the best temperature regulating performance. The temperature regulating article 100 can also be stored at cold temperature or high temperature environment before the use of the temperature regulating article 100 to prolong temperature regulating durability when the purpose of decreasing or increasing temperature is expected.

Thermal Buffer Tests

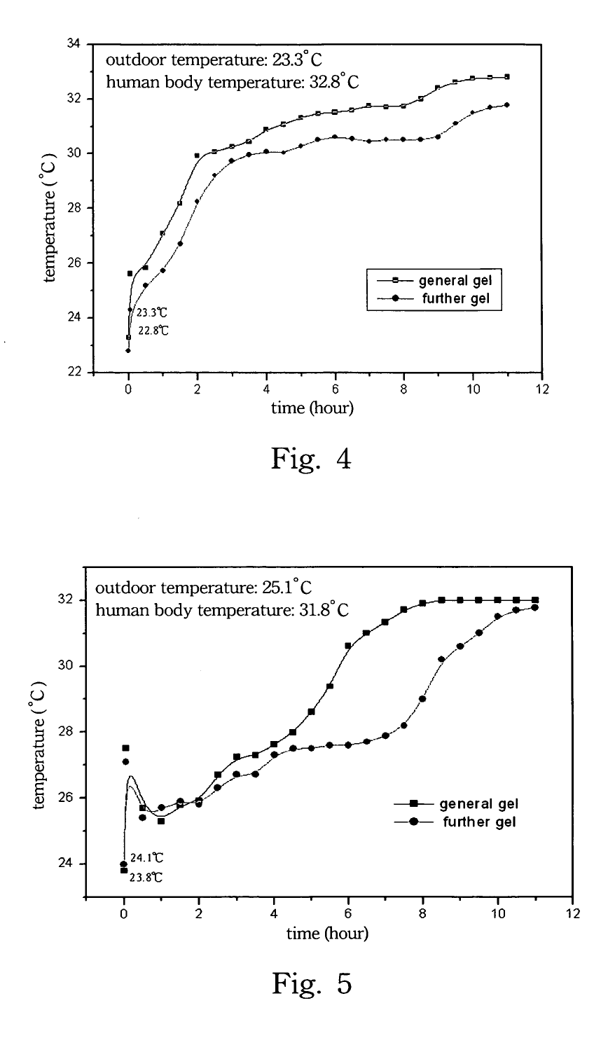

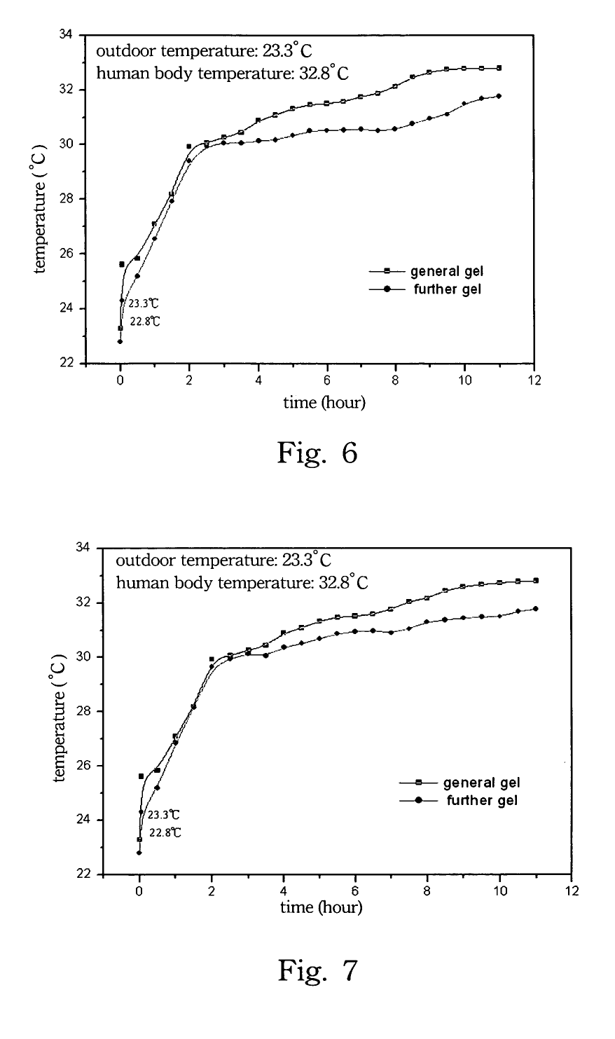

FIG. 4-7 are comparative results of thermal buffer tests for sample 1-4. The purpose of the temperature regulating gel in these tests was to lower down temperature. The “general gel” shown on the figures was the gel mixture of natural hydrogel and synthetic hydrogel and used as the comparative group.

The “further gel” used as the test sample was actually the same as the comparative gel mixture but added by 0.5 wt. % heat-storing material. The compositions of the general gel and further gel are listed in Table 1. The carboxylic ester heat-storing materials were used in samples 1-4 are listed in Table 2.

TABLE 1
sample and comparative group compositions

| | |
|---|---|
| sample | comparative |
| group | group |

| | |
|---|---|
| composition | (wt. %) |

| | | |
|---|---|---|
| carboxylic ester | 0.5 | — |
| heat-storing material | | |
| chitosan | 0.5 | 0.5 |
| gelatin | 0.5 | 0.5 |
| cross-linked polyethylene | 1.0 | 1.0 |
| glycol | | |
| polyvinylpyrrolidone | 2.0 | 2.0 |
| sodium carboxymethyl | 2.5 | 2.5 |
| cellulose | | |
| glycerine | 25.0 | 25.0 |
| deionized water | 68.0 | 68.0 |

TABLE 2
carboxylic ester heat-storing materials and
testing temperatures in samples 1-4.

| | | | carboxylic ester | |
|---|---|---|---|---|
| | human body | outdoor | heat-storing material | |
| | temperature | temperature | melting | molecular |
| | (° C.) | (° C.) | point (° C.) | weight |
| sample 1 | 32.8 | 23.3 | 24 | 284.5 |
| sample 2 | 31.8 | 25.1 | 28 | 326.5 |
| sample 3 | 32.8 | 23.3 | 30 | 312.5 |
| sample 4 | 32.8 | 23.3 | 37 | 298.5 |

Please refer to FIGS. 4, 6, and 7, outdoor temperature was 23.3° C. and human body temperature was 32.8° C. in these thermal buffer tests. Further gel and general gel had the same composition except general gel has 0.5 wt. % more carboxylic ester heat-storing material. The sample temperature-rising curves were below comparative temperature-rising curves revealed sample temperature-rising rate was smaller than that of the comparative group. Comparative temperatures were very close to human body temperatures after 9 hours. Sample temperatures were still lower than human body temperatures even after 11 hours which revealed better thermal buffer ability for sample group. In addition, the lower melting point of the carboxylic ester heat-storing material, the earlier separation between the sample temperature-rising curve and the comparative temperature-rising curve. But the sample temperature-rising curve and the comparative temperature-rising curve were very similar at the final stage.

FIG. 5 shows lower human body temperature 31.8° C. and higher outdoor temperature 25.1° C. The melting point of the carboxylic ester heat-storing material is 28° C. In FIG. 5, human body temperature is closer to outdoor temperature, so sample has better thermal buffer ability which leads to larger separation between the sample temperature-rising curve and the comparative temperature-rising curve than those in FIGS. 4, 6, and 7.

Accordingly, the temperature regulating article comprising the temperature regulating gel in the invention has long-lasting and stable temperature regulation and no additional controlling device is required. Furthermore, the temperature regulating article of this invention is portable, reusable and environmental protecting.

The temperature regulating gel of the invention has multiple industrial applications such as textile industrial, biomedical industrial, medical instrument industry, and cosmetic industry. The long-lasting temperature regulating gel can be essential material for moisturizing and temperature cooling applications such as wound cover and face mask. Furthermore, applications can be extended to health caring textile after combining with heat-storing material, or extended to other health caring products such as cosmetics and medical dressing after combining with Chinese herbal extracts.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A temperature regulating gel, comprising:
a hydrophilic polymeric substrate, the material of the hydrophilic polymeric substrate includes natural hydrogel, synthetic hydrogel, or any combination thereof;
a heat-storing material, dispersed in the hydrophilic polymeric substrate, the heat-storing material includes a phase change material, a plastic crystal, or any combination thereof; and
water.

2. The temperature regulating gel of claim 1, wherein the amount of the heat-storing material is at least 0.5% by weight.

3. The temperature regulating gel of claim 1, wherein the heat-storing material can be in the form of microcapsule.

4. The temperature regulating gel of claim 1, wherein the water content is around 30-90% by weight.

5. The temperature regulating gel of claim 1, wherein the natural gel includes anionic polymer, cationic polymer, amphoteric polymer, neutral polymer, or any combination thereof.

6. The temperature regulating gel of claim 5, wherein the anionic polymer can be hyaluronic acid, alginic acid, alginic acid derivative, pectin, chondroitin sulfate, gum arabic, carrageenan, lakh, xanthan gum, cellulose and cellulose derivative, or any combination thereof.

7. The temperature regulating gel of claim 5, wherein the cationic polymer can be chitosan, polylysine, or any combination thereof.

8. The temperature regulating gel of claim 5, wherein the amphoteric polymer can be collagen, gelatin, fibrin, or any combination thereof.

9. The temperature regulating gel of claim 5, wherein the neutral polymer can be dextran, agarose, starch and starch derivative, or any combination thereof.

10. The temperature regulating gel of claim 1, wherein the synthetic hydrogel includes non-cross-linked or cross-linked polymer.

11. The temperature regulating gel of claim 1, wherein the synthetic hydrogel includes polyethylene glycol and polyethylene glycol derivative, poly (acrylic acid), poly (acrylic acid) derivative, polyacrylamide, polyethacylamide, polyethacylamide derivative, polyvinylpyrrolidone, polyvinylpyrrolidone derivative, cellulose, cellulose derivative, silicone, silicone derivative, inorganic silicate, inorganic silicate derivative, magnesium aluminum silicate, modified natural hydrogel, or any copolymer of combination thereof.

12. The temperature regulating gel of claim 1, wherein the phase change material includes hydrocarbon, hydrate, wax, oil, fatty acid, fatty acid ester, carboxylic acid ester, dibasic esters, 1-halide, primary alcohol, aromatic compound, clathrate compound, semi-clathrate compound, gaseous clathrae compound, stearic anhydride, ethylene carbonate, polyol, polymer, metal, or any combination thereof.

13. The temperature regulating gel of claim 1, wherein the plastic crystal includes neopentyl glycol or neopentyl glycol derivative.

14. The temperature regulating gel of claim 1, further comprising an additive, the additive includes medicine, cosmetic material, dye, pigment,enzyme, spice, moisturizer, preservative, bactericide, bacteriostat, excipient, or any combination thereof.

15. A temperature regulating article, comprising:
a bottom layer; and
a gel layer, located on the bottom layer, comprising:
a hydrophilic polymeric substrate, comprising a natural hydrogel, a synthetic hydrogel, or any combination thereof,
a heat-storing material, dispersed in the hydrophilic polymeric substrate, the heat-storing material includes a phase change material, a plastic crystal, or any combination thereof; and
water.

16. The temperature regulating article of claim 15, wherein the amount of the heat-storing material is at least 0.5 wt. %.

17. The temperature regulating article of claim 15, wherein the heat-storing material can be in the form of microcapsule.

18. The temperature regulating article of claim 15, wherein the water content is around 30-90% by weight.

19. The temperature regulating article of claim 15, wherein the bottom layer includes thin film, woven fabric, nonwoven fabric, synthetic leather, animal leather, polyurethane, polyethylene, polypropylene, polyester, Teflon, or any combination thereof.

20. The temperature regulating article of claim 15, wherein the material of the bottom layer is thermal conducting material.

21. The temperature regulating article of claim 20, wherein the thermal conducting material is metal or metallic composite material, non-metal or non-metal composite material, or any combination thereof.

22. The temperature regulating article of claim 21, wherein the metal can be Al, Ag, Ti, Cu, Au, Sn, stainless steel, or Pb.

23. The temperature regulating article of claim 21, wherein the non-metal can be graphite, quartz, borosilicate glass, or polymer.

24. The temperature regulating article of claim 15, wherein an adhesive layer is between the bottom layer and the gel layer

25. The temperature regulating article of claim 15, wherein an adhesive layer is below the bottom layer

26. The temperature regulating article of claim 15, wherein a top layer is on the gel layer.

27. The temperature regulating article of claim 26, wherein the top layer includes thin film, woven fabric, nonwoven fabric, synthetic leather, animal leather, polyurethane, polyethylene, polypropylene, polyester, Teflon, or any combination thereof.

28. The temperature regulating article of claim 26, wherein the material of the bottom layer is thermal conducting material.

29. The temperature regulating article of claim 28, wherein the thermal conducting material is metal or metallic composite material, non-metal or non-metal composite material, or any combination thereof.

30. The temperature regulating article of claim 29, wherein the metal can be Al, Ag, Ti, Cu, Au, Sn, stainless steel, or Pb.

31. The temperature regulating article of claim 29, wherein the non-metal can be graphite, quartz, borosilicate glass, or polymer.

32. The temperature regulating article of claim 26, wherein the bottom layer and the top layer can be sealed to form a pocket like structure to have the gel layer contained in the pocket.

33. The temperature regulating article of claim 15, wherein the natural gel includes anionic polymer, cationic polymer, amphiphilic polymer, neutral polymer, or any combination thereof.

34. The temperature regulating article of claim 33, wherein the anionic polymer can be hyaluronic acid, alginic acid, alginic acid derivative, pectin, chondroitin sulfate, gum arabic, carrageenan, lakh, xanthan gum, cellulose and cellulose derivatives, or any combination thereof.

35. The temperature regulating article of claim 33, wherein the cationic polymer can be chitosan, polylysine, or any combination thereof.

36. The temperature regulating article of claim 33, wherein the amphiphilic polymer can be collagen, gelatin, fibrin, or any combination thereof.

37. The temperature regulating article of claim 33, wherein the neutral polymer can be dextran, agarose, starch and starch derivative, or any combination thereof.

38. The temperature regulating article of claim 15, wherein the synthetic hydrogel includes non-cross-linked or cross-linked polymer.

39. The temperature regulating article of claim 15, wherein the synthetic hydrogel includes polyethylene glycol and polyethylene glycol derivative, poly (acrylic acid), poly (acrylic acid) derivative, polyacrylamide, polyethacylamide, polyethacylamide derivative, polyvinylpyrrolidone, polyvinylpyrrolidone derivative, cellulose, cellulose derivative, silicone, silicone derivative, inorganic silicate, inorganic silicate derivative, magnesium aluminum silicate, modified natural hydrogel, or any copolymer of combination thereof.

40. The temperature regulating article of claim 15, wherein the phase change material includes hydrocarbon, hydrate, wax, oil, fatty acid, fatty acid ester, carboxylic acid ester, dibasic esters, 1-halide, primary alcohol, aromatic compound, clathrate compound, semi-clathrate compound, gaseous clathrae compound, stearic anhydride, ethylene carbonate, polyol, polymer, metal, or any combination thereof.

41. The temperature regulating article of claim 15, wherein the plastic crystal includes neopentyl glycol or neopentyl glycol derivatives.

42. The temperature regulating article of claim 15, further comprising an additive, the additive includes medicine, cosmetic material, dye, pigment, enzyme, spice, moisturizer, preservative, bactericide, bacteriostat, excipient, or any combination thereof.
